(19) United States
(12) Patent Application Publication
(10) Pub. No.: US 2023/0052546 A1
(43) Pub. Date: Feb. 16, 2023

(54) HAND SANITIZER MICROEMULSION

(71) Applicant: Fluid Energy Group Ltd., Calgary (CA)

(72) Inventors: Elsayed ABDELFATAH, Calgary (CA); Markus WEISSENBERGER, Calgary (CA)

(21) Appl. No.: 17/877,701

(22) Filed: Jul. 29, 2022

(30) Foreign Application Priority Data
Jul. 30, 2021 (CA) 3126520

(51) Int. Cl. A61K 8/06; A61Q 17/00; A61K 8/34

(52) U.S. Cl. CPC A61K 8/068; A61Q 17/005; A61K 8/34; A61K 2800/5424

(57) ABSTRACT

The present disclosure describes a method of recycling an expired hand sanitizer product containing ethanol in an amount ranging from 60 to 85 wt %, wherein said method comprises the steps of: providing said hand sanitizer product; combining said hand sanitizer product with an anionic surfactant; adding a nonionic surfactant to the resulting mixture; and adding citral to the mixture containing said hand sanitizer, nonionic surfactant and said anionic surfactant to create a stable microemulsion; wherein said stable microemulsion can be further diluted with an acidic composition to be used in industrial applications comprising: flowback aids for hydraulic fracturing in tight reservoirs and unconventional shale reservoirs; naphthenate deposition control using acids/alcohols mixtures; or water-in-oil demulsifier using ethanol for bitumen emulsions from oilsand.

### CROSS-REFERENCE TO RELATED APPLICATION

This application claims the benefit of Canadian Patent Application No. 3,126,520 filed Jul. 30, 2021, the entire contents of which are incorporated herein by reference in its entirety.

### FIELD OF THE INVENTION

The present invention is directed to the upcycling of expired hand sanitizer solutions, more specifically, it is directed to the use of expired hand sanitizer solutions in industrial applications.

### BACKGROUND OF THE INVENTION

After the COVID-19 pandemic, it has been decided that the Federal government of Canada will maintain a national reserve of hand sanitizer for potential future emergency use.

Hand sanitizers like many other products have a set lifetime for safe use, beyond this lifetime, it is recommended to no longer use such products and it is highly recommended to discard any remaining product. Given the difficulty of predicting spikes in uses by the population, or the need for stockpiling, it is desirable to develop an alternative approach where expired hand sanitizer can be recycled into industrially useful products.

Accordingly, industrial applications, like oilfield and industrial cleaners have been considered to absorb expired hand sanitizer and incorporate such into useful operations. In certain settings, the upcycled hand sanitizers can replace chemicals such as butyl carbitol and since the products being upcycled will be extremely cheap to acquire or even being paid to recycled such, they will be price competitive with any other compound on the market which is not being upcycled.

Hand Sanitizer (HS) effective disinfectant against viruses like COVID-19, must contain compounds such as ethanol or isopropanol. In one embodiment of the present invention, a hand sanitizer comprising 80% USP ethanol; hydrogen peroxide (H₂O₂); glycerol; and water.

Several applications in the oilfield have been identified as potential targets for the upcycling of hand sanitizer, these include but are not limited to: flowback aids for hydraulic fracturing in tight reservoirs and unconventional shale reservoirs; naphthenate deposition control using acids/alcohols mixtures; and water-in-oil demulsifier using ethanol for bitumen emulsions from oilsand.

Flowback Aids for Hydraulic Fracturing in Tight Reservoirs and Unconventional Shale Reservoirs

Hydraulic fracturing is the most widely used well stimulation technique especially for tight reservoirs and shale gas reservoirs. A key issue with hydraulic fracturing is the formation damage caused by the frac fluid invasion into the formation matrix and the formation of oil/water emulsions. Microemulsions were developed as a flowback additive for tight gas reservoirs. Microemulsions can encapsulate the surfactants to slow the adsorption so they can penetrate deeper into the formation to restore the permeability to gas. Microemulsions are formed using mixed surfactant systems and short-chain alcohol like ethanol used in the hand sanitizer.

Naphthenate Deposition Control Using Acids/Alcohols Mixtures

Metal naphthenates stabilize oil-in-water emulsions and in some cases form highly viscous sludge. Mineral acids have been proven to be effective in solving the naphthenate scale problems. However, mineral acids, like HCl and acetic acids, are corrosive and associated with significant HSE hazardous. Fluid Energy Group Ltd. proprietary modified acid Enviro-Syn HCR solves a large number of the operational and HSE issues associated with HCl without affecting performance. Mutual solvents like ethanol in hand sanitizer can improve the solvency and the dissolution of naphthenate soaps in the acid.

Water-in-oil Demulsifier Using Ethanol for Bitumen Emulsions from Oilsand

Water-in-Oil (W/O) emulsions are the most common issue in the oilfield, especially in bitumen production from oilsands. Demulsifying W/O emulsions has a significant environmental impact; recovering the water with high quality for recycling, and minimizing oil transportation cost and environmental footprint. Oil-soluble demulsifiers like mixed aromatics/low-alcohols (like ethanol in hand sanitizer) are commonly used for demulsifying W/O emulsions. Water-soluble demulsifiers can be made oil-soluble by adding a coupling solvent such as short-chain alcohols (like ethanol in hand sanitizer). Microemulsion-based demulsifiers can enhance the wellbore cleaning when used downhole.

In light of the worldwide situation, it is desirable to have a stockpile of hand sanitizer but also an efficient method to handle expired products and not cause an environmental disaster by having to discard large volumes of hand sanitizer. To date, no solution has been proposed capable of handling large volumes of expired products. In light of this, there is a clear need to be able to handle expired products without causing environmental damage by upcycling, or reusing such.

### SUMMARY OF THE INVENTION

According to a first aspect of the present invention, there is provided a microemulsion composition comprising:
a hand sanitizer product in an amount ranging from 10 to 40 wt % of the total weight of the composition, said hand sanitizer product comprising ethanol in an amount ranging from 60 to 85 wt %;
oil phase in an amount ranging from 10 to 40 wt % of the total weight of the composition;
a nonionic surfactant present in an amount ranging from 10 to 30 wt % of the total weight of the composition; and
an anionic surfactant present in an amount ranging from 5 to 15 wt % of the total weight of the composition.

According to a preferred embodiment of the present invention, the oil phase is present in an amount ranging from 20 to 35 wt % of the total weight of the composition. Preferably, the citral is present in an amount ranging from 25 to 30 wt % of the total weight of the composition.

According to a preferred embodiment of the present invention, the nonionic surfactant present in an amount ranging from 15 to 25 wt % of the total weight of the composition. Preferably, the nonionic surfactant present in an amount of approximately 20 wt % of the total weight of the composition.

According to a preferred embodiment of the present invention, the anionic surfactant present in an amount ranging from 7 to 13 wt %. Preferably, the anionic surfactant present in an amount of approximately 10 wt % of the total weight of the composition.

According to a preferred embodiment of the present invention, the oil phase is selected from the group consisting of: citral, terpenes, hydrocarbon oils, or methyl esters. Preferably, the oil phase is Citral.

According to a preferred embodiment of the present invention, the anionic surfactant is selected from the group consisting of: anionic sulfonate surfactant. Preferably, the anionic surfactant is selected from the group consisting of: Dodecyl benzene sulfonic acid (DDBSA); Alkyldiphenyloxide Disulfonate (Dowfax® C10L); and combinations thereof.

According to a preferred embodiment of the present invention, the nonionic surfactant is selected from the group consisting of nonionic linear or branched alcohol ethoxylate. Preferably, the nonionic surfactant is selected from the group consisting of: Novel® 23E3; Novel® 23E7; Lutensol® XL90; and combinations thereof.

According to another aspect of the present invention, there is provided a method of upcycling an expired hand sanitizer product containing ethanol in an amount ranging from 60 to 85 wt %, wherein said method comprises the steps of:
providing said hand sanitizer product;
combining said hand sanitizer product with an anionic surfactant;
adding a nonionic surfactant to the resulting mixture;
adding oil phase to the mixture containing said hand sanitizer, nonionic surfactant, and said anionic surfactant to create a stable microemulsion;
   wherein said stable microemulsion can be further diluted with an acidic composition to be used in industrial applications comprising:
flowback aids for hydraulic fracturing in tight reservoirs and unconventional shale reservoirs;
naphthenate deposition control using acids/alcohols mixtures;
water-in-oil demulsifier using ethanol for bitumen emulsions from oilsand.

According to another aspect of the present invention, there is provided a use of microemulsion in combination with a modified acid microemulsion-based dissolver to remove of mixed scale deposition.

According to yet another aspect of the present invention, there is provided a use of a microemulsion in combination with a modified acid as a microemulsion-based dissolver for cleaning of surface facilities.

According to yet another aspect of the present invention, there is provided a use of a microemulsion in combination with a modified acid as a microemulsion-based dissolver cleanup of wellbore deposits concentrated with paraffin and asphaltene.

According to yet another aspect of the present invention, there is provided a use of a microemulsion in combination with a modified acid microemulsion-based dissolver for filter cake removal.

### BRIEF DESCRIPTION OF THE FIGURES

Features and advantages of embodiments of the present application will become apparent from the following detailed description and the appended figures, in which:

FIG. 1 graphical representation of the particle size distribution from dynamic light scattering measurement for composition HS-ME3;

FIG. 2 graphical representation of the particle size distribution from dynamic light scattering measurement for composition HS-ME4; and

FIG. 3 graphical representation of the particle size distribution from dynamic light scattering measurement for composition HS-ME5.

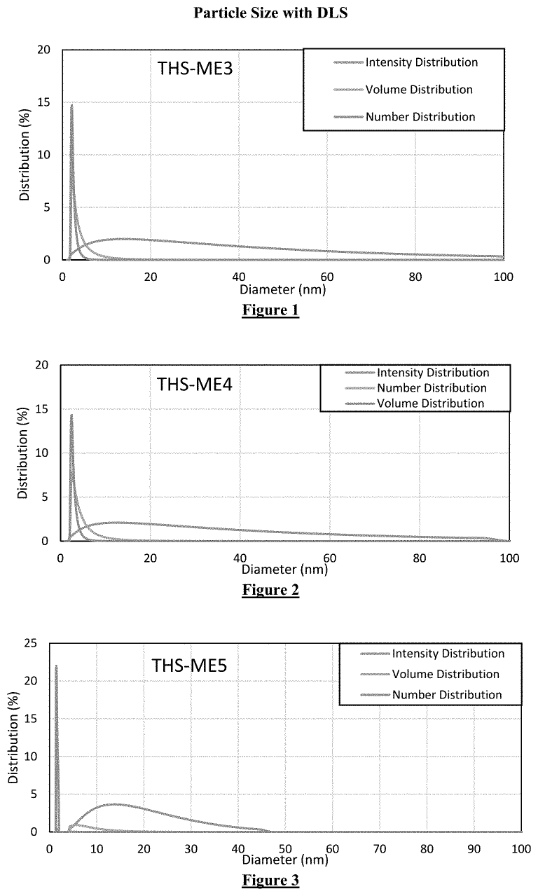

### DESCRIPTION OF PREFERRED EMBODIMENTS OF THE PRESENT INVENTION

It will be appreciated that numerous specific details have been provided for a thorough understanding of the exemplary embodiments described herein. However, it will be understood by those of ordinary skill in the art that the embodiments described herein may be practiced without these specific details. In other instances, well-known methods, procedures, and components have not been described in detail so as not to obscure the embodiments described herein. Furthermore, this description is not to be considered so that it may limit the scope of the embodiments described herein in any way, but rather as merely describing the implementation of the various embodiments described herein.

Microemulsions were formed using nonionic surfactant (Novel® 23E7, Novel® 23E3, Lutensol® XL90) and anionic sulfonate surfactant (Dodecyl Benzene Sulfonic Acid (DDBSA) or DOWFAX® C10L). Hand sanitizer is tested as a cosolvent and compared to butyl carbitol. Citral and mineral oil were studied as components of the oil phase.

### Preparation of the Microemulsion

Each component of the formulation was added sequentially and thoroughly mixed before adding the next component. Finally, the oil component was added gradually while mixing to find the maximum dissolution capacity of the formulation.

### Dilution of the Microemulsion

The microemulsion is then diluted with several solutions (purified water, 2% KCl solutions, Acid composition #1; Acid composition #2; Acid composition #3; or Acid composition #4 with a loading of 2 gpt (0.2%). The resulting dilutions were observed visually and recorded.

Composition #1 was prepared as per the following. Monoethanolamine (MEA) and hydrochloric acid are used as starting reagents. To obtain a 4.1:1 molar ratio of MEA to HCl, one must first mix 165 g of MEA with 835 g of water. This forms the monoethanolamine solution. Subsequently, one takes 370 ml of the previously prepared monoethanolamine solution and mixes with 350 ml of HCl aq. 36% (22 Baume). In the event that additives are used, they are added after thorough mixing of the MEA solution and HCl. For example, potassium iodide can be added at this point as well as any other component desired to optimize the performance of the composition according to the present invention. Circulation is maintained until all products have been solubilized. Additional products can now be added as required. The resulting composition of Example 1 is a clear (slightly yellow) liquid having shelf-life of greater than 1 year. It has a boiling point temperature of approximately 100 C. It has a specific gravity of 1.1 0.02. It is completely soluble in water and its pH is less than 1. The freezing point was determined to be less than −35 C. The organic component in the composition is biodegradable. The composition is classified as a mild irritant according to the classifications for skin tests.

Acid composition #1 comprises a blend of HCl with MEA in a 4.1:1 molar ratio.

Acid composition #2 comprises: 28% HCl with a CI package comprising: citral; B-Alanine, N-(2-carboxyethyl)-N-dodecyl-, sodium salt (1:1); cocamidopropyl betaine; propargyl alcohol complexed with methyloxirane; potassium iodide; Novel® 23E7; and ethanol.

Acid composition #3 comprises a blend of HCl with MEA in a 6.4:1 molar ratio with a citral; B-Alanine, N-(2-carboxyethyl)-N-dodecyl-, sodium salt (1:1); cocamidopropyl betaine; propargyl alcohol complexed with methyloxirane; potassium iodide; Novel® 23E7; and isopropanol. Acid composition #3 was made by the similar methodology as acid composition #1 but with the difference in the HCl:MEA ratio and the incorporation of a CI package.

Acid composition #4 comprises a blend of HCl with MEA in a 6.4:1 molar ratio with a citral; B-Alanine, N-(2-carboxyethyl)-N-dodecyl-, sodium salt (1:1); cocamidopropyl betaine; propargyl alcohol complexed with methyloxirane; potassium iodide; Novel® 23E7; and ethanol. Acid composition #4 was made by the similar methodology as acid composition #1 but with the difference in the HCl:MEA ratio and the incorporation of a CI package.

### Testing with 30 wt % Oil (Citral)

Nonionic/Anionic Surfactant Mixture

Formulations were made with a constant concentration and ratio of both surfactants; 20 wt % nonionic surfactant (Novel® 23E7) and 10 wt % anionic surfactants (DDBSA or Dowfax® C10L).

In a first series of tests, hand sanitizer was combined with the nonionic surfactant (Novel® 23E7) and the anionic surfactant DDBSA. The results and formulations of compositions labelled HS-ME11, HS-ME12, HS-ME13, HS-ME14, and HS-ME15 are reported in Table 1 below. All compositions except for HS-ME11 formed a clear solution.

Mineral oil was studied. Although it dissolved and formed microemulsion, it did not have good solubility capacity in the formulation as citral. Hence, citral was tested for the rest of the formulations and shown good solubility up to 30 wt % (maximum tested). The preparation of the microemulsion involved the gradual addition of oil until the entire pre-determined amount is added or until the solution becomes turbid, at which point no more oil was added.

It was noted that solubilization capacity increases with increasing the concentration of co-solvents.

In the tables below the following legend applies:
Y=Yes, amount of oil was added
X=No, amount of oil was not added
C=Clear solution
T=Turbid solution
V=Viscous
VV=Very Viscous
H=Heat applied

Turbid microemulsions show phase separation into two clear solutions overnight with free oil on the top of O/W emulsion. Turbid diluted microemulsion in base fluids shows phase separation after staying quiescent.

TABLE 1
Formulations containing citral and a varying amount of recycled hand
sanitizer and DDBSA and Novel ® 23E7 as surfactant mixture and comments
on the visual aspect of each prepared composition

| | HS-ME11 | | HS-ME12 | | HS-ME13 | | HS-ME14 | | HS-ME15 | |
|---|---|---|---|---|---|---|---|---|---|---|
| | wt % | g | wt % | g | wt % | g | wt % | g | wt % | g |
| Water | 35.0 | 5.3 | 30.0 | 4.5 | 25.0 | 3.8 | 20.0 | 3.0 | 15.0 | 2.3 |
| Hand sanitizer | 5.0 | 0.8 | 10.0 | 1.5 | 15.0 | 2.3 | 20.0 | 3.0 | 25.0 | 3.8 |
| DDBSA | 10.0 | 1.5 | 10.0 | 1.5 | 10.0 | 1.5 | 10.0 | 1.5 | 10.0 | 1.5 |
| Novel ® 23E7 | 20.0 | 3.0 | 20.0 | 3.0 | 20.0 | 3.0 | 20.0 | 3.0 | 20.0 | 3.0 |
| Citral | 30.0 | 4.5 | 30.0 | 4.5 | 30.0 | 4.5 | 30.0 | 4.5 | 30.0 | 4.5 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.90 | Y | T/V | Y | C | Y | C | Y | C | Y | C |
| 1.80 | X | | Y | C | Y | C | Y | C | Y | C |
| 2.70 | | | Y | C | Y | C | Y | C | Y | C |
| 3.60 | | | Y | C | Y | C | Y | C | Y | C |
| 4.50 | | | Y | C | Y | C | Y | C | Y | C |

In a second series of tests, hand sanitizer was combined with the nonionic surfactant (Novel® 23E7) and the anionic surfactant Dowfax® C10L. The results and formulations of compositions labelled HS-ME16, HS-ME17, HS-ME18, HS-ME19 and HS-ME20 are reported in Table 2 below. Only compositions labelled HS-ME19 and HS-ME-20 formed a clear solution with the full amount of oil component (citral) added in the composition.

TABLE 2
Formulations containing citral and a varying amount of recycled hand
sanitizer and DOWFAX ® C10L and Novel ® 23E7 as surfactant mixture and
comments on the visual aspect of each prepared composition

| | HS-ME16 | | HS-ME17 | | HS-ME18 | | HS-ME19 | | HS-ME20 | |
|---|---|---|---|---|---|---|---|---|---|---|
| | wt % | g | wt % | g | wt % | g | wt % | g | wt % | g |
| Water | 35.0 | 5.25 | 30.0 | 4.5 | 25.0 | 3.75 | 20.0 | 3 | 15.0 | 2.25 |
| Hand sanitizer | 5.0 | 0.75 | 10.0 | 1.5 | 15.0 | 2.25 | 20.0 | 3 | 25.0 | 3.75 |
| DOWFAX ® C10L | 10.0 | 1.5 | 10.0 | 1.5 | 10.0 | 1.5 | 10.0 | 1.5 | 10.0 | 1.5 |
| Novel ® 23E7 | 20.0 | 3 | 20.0 | 3 | 20.0 | 3 | 20.0 | 3 | 20.0 | 3 |
| Citral | 30.0 | 4.5 | 30.0 | 4.5 | 30.0 | 4.5 | 30. | 4.5 | 30.0 | 4.5 |
| Total | 100.0 | 15 | 100.0 | 15 | 100.0 | 15 | 100.0 | 15 | 100.0 | 15 |
| Citral (g) | | | | | | | | | | |
| 0.90 | Y | C | Y | C | Y | C | Y | C | Y | C |
| 1.80 | Y | T | Y | C | Y | C | Y | C | Y | C |
| 2.70 | X | | Y | T | Y | C | Y | C | Y | C |
| 3.60 | | | | | Y | T | Y | C | Y | C |
| 4.50 | | | | | | | Y | C | Y | C |

In a third series of tests, hand sanitizer was replaced with butyl carbitol and this was combined with the nonionic surfactant (Novel® 23E7) and the anionic surfactant DDBSA. The results and formulations of compositions labelled HS-ME21, HS-ME22, HS-ME23, HS-ME24 and HS-ME25 are reported in Table 3 below. Only compositions labelled HS-ME21 did not form a clear solution with the full amount of oil component (citral) added in the composition.

TABLE 3
Formulations containing citral and a varying amount of butyl carbitol and
DDBSA and Novel 23E7 as surfactant mixture and comments on the visual
aspect of each prepared composition

| | HS-ME21 | | HS-ME22 | | HS-ME23 | | HS-ME24 | | HS-ME25 | |
|---|---|---|---|---|---|---|---|---|---|---|
| | wt % | g | wt % | g | wt % | g | wt % | g | wt % | g |
| Water | 35.0 | 5.3 | 30.0 | 4.5 | 25.0 | 3.8 | 20.0 | 3.0 | 15.0 | 2.3 |
| Butyl Carbitol | 5.0 | 0.8 | 10.0 | 1.5 | 15.0 | 2.3 | 20.0 | 3.0 | 25.0 | 3.8 |
| DDBSA | 10.0 | 1.5 | 10.0 | 1.5 | 10.0 | 1.5 | 10.0 | 1.5 | 10.0 | 1.5 |
| Novel ® 23E7 | 20.0 | 3.0 | 20.0 | 3.0 | 20.0 | 3.0 | 20.0 | 3.0 | 20.0 | 3.0 |
| Citral | 30.0 | 4.5 | 30.0 | 4.5 | 30.0 | 4.5 | 30.0 | 4.5 | 30.0 | 4.5 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.90 | Y | T | Y | C | Y | C | Y | C | Y | C |
| 1.80 | X | | Y | C | Y | C | Y | C | Y | C |
| 2.70 | | | Y | C | Y | C | Y | C | Y | C |
| 3.60 | | | Y | C | Y | C | Y | C | Y | C |
| 4.50 | | | Y | C | Y | C | Y | C | Y | C |

In a fourth series of tests, butyl carbitol was combined with the nonionic surfactant (Novel® 23E7) and the anionic surfactant Dowfax® C10L. The results and formulations of compositions labelled HS-ME26, HS-ME27, HS-ME28, HS-ME29 and HS-ME30 are reported in Table 4 below. Compositions labelled HS-ME26 and HS-ME-27 did not form a clear solution with the full amount of oil component (citral) added in the composition.

TABLE 4
Formulations containing citral and a varying amount of butyl carbitol and
DOWFAX ^{®} C10L and Novel ^{®} 23E7 as surfactant mixture and comments on
the visual aspect of each prepared composition

| | HS-ME26 | | HS-ME27 | | HS-ME28 | | HS-ME29 | | HS-ME30 | |
|---|---|---|---|---|---|---|---|---|---|---|
| | wt % | g | wt % | g | wt % | g | wt % | g | wt % | g |
| Water | 35.0 | 5.3 | 30.0 | 4.5 | 25.0 | 3.8 | 20.0 | 3.0 | 15.0 | 2.3 |
| Butyl Carbitol | 5.0 | 0.8 | 10.0 | 1.5 | 15.0 | 2.3 | 20.0 | 3.0 | 25.0 | 3.8 |
| DOWFAX ^{®} C10L | 10.0 | 1.5 | 10.0 | 1.5 | 10.0 | 1.5 | 10.0 | 1.5 | 10.0 | 1.5 |
| Novel ^{®} 23E7 | 20.0 | 3.0 | 20.0 | 3.0 | 20.0 | 3.0 | 20.0 | 3.0 | 20.0 | 3.0 |
| Citral | 30.0 | 4.5 | 30.0 | 4.5 | 30.0 | 4.5 | 30.0 | 4.5 | 30.0 | 4.5 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.90 | X | | Y | C | Y | C | Y | C | Y | C |
| 1.80 | | | Y | C | Y | C | Y | C | Y | C |
| 2.70 | | | Y | C | Y | C | Y | C | Y | C |
| 3.60 | | | Y | T | Y | C | Y | C | Y | C |
| 4.50 | | | | | Y | C | Y | C | Y | C |

Dilutions

Photographs were taken of compositions HS-ME15, HS-ME20, HS-ME25 and HS-ME30 (diluted to 2 gpt) in 2% KCl solution. Formulations with Dowfax® C10L are salt-tolerant and form clear solutions when diluted. There was no major difference between the compositions containing hand sanitizer and those containing butyl carbitol. The visual appearance did not change for a few days. Turbid formulations did not show any separation after a week.

Photographs taken of compositions HS-ME15, HS-ME 20, HS-ME25 and HS-ME30 diluted in acid composition #4 and acid composition #1 show that the microemulsions are turbid when diluted with acid composition #1. The microemulsions do not change the color or turbidity of acid composition #4. The corrosion inhibitor (CI) package in acid composition #4 is believed to aid in enhancing the stability of the microemulsion.

When composition labelled HS-ME15 was diluted in acid composition #1 it exhibited some separation of DDBSA of a few days. When composition labelled HS-ME30 was diluted in acid composition #4 it exhibited black separation on the top after 1 day. None of the other samples showed any notable change in visual appearance over time.

None of the compositions were stable in acid composition #1 as phase separation was observed. Meanwhile, in acid composition #4, the formulations were stable after a few days and did not show any separation after a few days.

### Surface Tension Measurements

The surface tension (SFT) was measured using a Wilhelmy plate with a Kruss 100C force tensiometer for compositions HS-ME15, HS-ME 20, HS-ME25 and HS-ME30.

### Dynamic Contact Angle Measurements

Dynamic contact angle measurements were conducted using the Wilhelmy plate method with a Kruss 100C® force tensiometer. The results are reported in Tables 5 and 6 below. A parafilm plate was used as a hydrophobic surface to measure the efficiency of the formulations in reducing the contact angles. The advancing and receding contact angles (θ_{A} and θ_{R}) were measured. The advancing and receding contact angles of parafilm with purified water are around 120 and 95, respectively. All the microemulsions are shown to significantly reduce the contact angle with a hydrophobic surface like parafilm. This is an indication of significant enhancement of the wetting character of the fluid and efficient application for wettability alteration in reservoir rocks.

TABLE #5
Surface tension and contact angle of diluted formulations
Diluted to ~0.2 vol % in 2% KCl Solution [0.1 mL + 49.9 mL]

| | SFT | ACA | RCA | |
|---|---|---|---|---|
| Formulation | (mN/m) | (Parafilm) | (Parafilm) | Visual |
| HS-ME15 | 29.17 | 56.78 | 5.22 | Turbid |
| HS-ME20 | 29.97 | 57.48 | 8.06 | Clear |
| HS-ME25 | 29.22 | 51.09 | 8.67 | Turbid |
| HS-ME30 | 30.07 | 55.91 | 9.55 | Clear |

TABLE #6
Surface tension and contact angle of formulations diluted to
~0.2 vol % in 2% KCl Solution [0.1 mL + 49.9 mL]
Dilution of HS-ME15 (2 gpt ~0.2 vol %) in HCR
[0.1 mL + 49.9 mL]

| | SFT | ACA | RCA | |
|---|---|---|---|---|
| | (mN/m) | (Parafilm) | (Parafilm) | Visual |
| acid | 31.48 | 65.38 | 8.60 | Initially green and then |
| composition | | | | back to the original |
| #4 | | | | color |
| acid | 31.14 | 62.46 | 5.88 | Immediately turbid |
| composition | | | | |
| #1 | | | | |

TABLE #7
Surface tension and contact angle of formulations diluted to
~0.2 vol % in various HCR solutions [0.1 mL + 49.9 mL]
Dilution of HS-ME20 (2 gpt ~0.2 vol %) in HCR
[0.1 mL + 49.9 mL]

| | SFT | ACA | RCA | |
|---|---|---|---|---|
| | (mN/m) | (Parafilm) | (Parafilm) | Visual |
| acid | 29.98 | 60.6 | 8.62 | The original color |
| composition | | | | did not change |
| #4 | | | | |
| acid | 29.31 | 55.8 | 0.74 | Initially clear then |
| composition | | | | changed to turbid |
| #1 | | | | over time |

### Effect of Neutralization of DDBSA with MEA (1:1 M)

MEA was added as a 1:1 M with DDBSA. The reaction was exothermic. However, it is evident that MEA reduced the effectiveness of the formula at 10 wt % hand sanitizer (HS). However, it did not show any change at higher concentrations of HS.

Compositions HS-ME33 and 35 were diluted in KCl solution and both resulted in turbid solutions.

TABLE 8
Formulations containing citral and a varying amount of recycled hand
sanitizer and MEA present as a neutralizing agent and DDBSA and Novel ^{®}
23E7 as surfactant mixture and comments on the visual aspect of each
prepared composition

| | HS-ME31 | | HS-ME32 | | HS-ME33 | | HS-ME34 | | HS-ME35 | |
|---|---|---|---|---|---|---|---|---|---|---|
| | wt % | g | wt % | g | wt % | g | wt % | g | wt % | g |
| Water | 33.2 | 4.98 | 28.2 | 4.23 | 23.2 | 3.48 | 18.2 | 2.73 | 13.2 | 1.98 |
| Hand | 5.0 | 0.75 | 10.0 | 1.50 | 15.0 | 2.25 | 20.0 | 3.00 | 25.0 | 3.75 |
| sanitizer | | | | | | | | | | |
| MEA | 1.8 | 0.27 | 1.8 | 0.27 | 1.8 | 0.27 | 1.8 | 0.27 | 1.8 | 0.27 |
| DDBSA | 10.0 | 1.50 | 10.0 | 1.50 | 10.0 | 1.50 | 10.0 | 1.50 | 10.0 | 1.50 |
| Novel ^{®} 23E7 | 20.0 | 3.00 | 20.0 | 3.00 | 20.0 | 3.00 | 20.0 | 3.00 | 20.0 | 3.00 |
| Citral | 30.0 | 4.50 | 30.0 | 4.50 | 30.0 | 4.50 | 30.0 | 4.50 | 30.0 | 4.50 |
| Total | 100.0 | 15.0 | 100.0 | 15.0 | 100.0 | 15.0 | 100.0 | 15.0 | 100.0 | 15.0 |
| Citral | | | | | | | | | | |
| 0.90 | Y | T/V | Y | C | Y | C | Y | C | C | C |
| 1.80 | | | Y | T/V | Y | C | Y | C | C | C |
| 2.70 | | | | | Y | C | Y | C | C | C |
| 3.60 | | | | | Y | C | Y | C | C | C |
| 4.50 | | | | | Y | C | Y | C | C | C |

### Testing with 15 wt % Oil (Citral)

Nonionic/Anionic Surfactant Mixture

A mixture of nonionic/anionic surfactants is tested for solubilization of 15 wt % Citral by varying the concentration of hand sanitizer. The anionic surfactant tested in this series was DDBSA and the nonionic surfactant was Novel® 23E7. As shown in Table 9, low hand sanitizer concentrations (5 and 10 wt %) could only solubilize 3 wt % citral. As the concentration of hand sanitizer increases to 15% and up to 25 wt %, all of the citral added was solubilized into clear transparent solutions.

A photograph was taken of the composition HS-ME5 (2 gpt) diluted in various media and resulted in the following: in DIW (the result of the dilution was a clear solution); in 2% KCl (the result of the dilution was a turbid solution); in acid composition #1 (the result of the dilution was a turbid solution); in acid composition #2 (the result of the dilution was a turbid solution); and in Acid composition #4 (the result of the dilution was a turbid solution similar to Acid composition #3).

Upon visual analysis of the above series of dilutions, it is clear that the surfactant mixture does not provide satisfactory stability for any of the tested dilution scenarios.

TABLE 9
Formulations containing citral and a varying amount of hand sanitizer using
DDBSA and Novel ^{®} 23E7 and comments on the visual aspect of each prepared
composition

| | HS-ME1 | | HS-ME2 | | HS-ME3 | | HS-ME4 | | HS-ME5 | |
|---|---|---|---|---|---|---|---|---|---|---|
| | wt % | g | wt % | g | wt % | g | wt % | g | wt % | g |
| Water | 50.0 | 7.5 | 45.0 | 6.8 | 40.0 | 6.0 | 35.0 | 5.3 | 30.0 | 4.5 |
| Hand sanitizer | 5.0 | 0.8 | 10.0 | 1.5 | 15.0 | 2.3 | 20.0 | 3.0 | 25.0 | 3.8 |
| DDBSA | 10.0 | 1.5 | 10.0 | 1.5 | 10.0 | 1.5 | 10.0 | 1.5 | 10.0 | 1.5 |
| Novel ^{®} 23E7 | 20.0 | 3.0 | 20.0 | 3.0 | 20.0 | 3.0 | 20.0 | 3.0 | 20.0 | 3.0 |
| Citral | 15.0 | 2.3 | 15.0 | 2.3 | 15.0 | 2.3 | 15.0 | 2.3 | 15.0 | 2.3 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | Y | C | Y | C | Y | C | Y | C | Y | C |
| 0.90 | Y | T | Y | T | Y | C | Y | C | Y | C |
| 1.35 | X | | | | Y | C | Y | C | Y | C |
| 1.80 | | | | | Y | C | Y | C | Y | C |
| 2.25 | | | | | Y | C | Y | C | Y | C |

Another series of testing investigated a mixture of nonionic/anionic surfactants for use in the solubilization of 15 wt % citral by varying the concentration of hand sanitizer. However, in this series of testing, the anionic surfactant is replaced with Dowfax® C10L. The nonionic surfactant is the same as before, Novel® 23E7. As shown in the table below, low hand sanitizer concentrations (5 wt %) cannot solubilize any citral because the surfactant solution is very viscous. However, unlike the case of DDBSA, 10 wt % hand sanitizer with Dowfax® C10L can solubilize up to 12 wt % citral. As the concentration of HS increases to 15% and even up to 25 wt %, all of the citral added into the composition can be solubilized into clear transparent solutions.

TABLE 10
Formulations containing citral and a varying amount of hand sanitizer using
Dowfax ^{®} C10L and Novel ^{®} 23E7 as surfactant mixture and comments on the
visual aspect of each prepared composition

| | HS-ME61 | | HS-ME62 | | HS-ME63 | | HS-ME64 | | HS-ME65 | |
|---|---|---|---|---|---|---|---|---|---|---|
| | wt % | g | wt % | g | wt % | g | wt % | g | wt % | g |
| Water | 50.0 | 7.5 | 45.0 | 6.8 | 40.0 | 6.0 | 35.0 | 5.3 | 30.0 | 4.5 |
| HS | 5.0 | 0.8 | 10.0 | 1.5 | 15.0 | 2.3 | 20.0 | 3.0 | 25.0 | 3.8 |
| DOWFAX ^{®} C10L | 10.0 | 1.5 | 10.0 | 1.5 | 10.0 | 1.5 | 10.0 | 1.5 | 10.0 | 1.5 |
| Novel ^{®} 23E7 | 20.0 | 3.0 | 20.0 | 3.0 | 20.0 | 3.0 | 20.0 | 3.0 | 20.0 | 3.0 |
| Citral | 15.0 | 2.3 | 15.0 | 2.3 | 15.0 | 2.3 | 15.0 | 2.3 | 15.0 | 2.3 |
| Total | 100.0 | 15.0 | 100.0 | 15.0 | 100.0 | 15.0 | 100.0 | 15.0 | 100.0 | 15.0 |
| Citral(g) | | | | | | | | | | |
| 0.45 | X | | Y | C | Y | C | Y | C | Y | C |
| 0.90 | | | Y | C | Y | C | Y | C | Y | C |
| 1.35 | | | Y | C | Y | C | Y | C | Y | C |
| 1.80 | | | Y | C | Y | C | Y | C | Y | C |
| 2.25 | | | Y | T | Y | C | Y | C | Y | C |

A photograph was taken of the composition HS-ME65 (2 gpt) diluted in various media and resulted in the following: in DIW, the result of this dilution was a clear solution; in 2% KCl: the result of this dilution was a clear solution; in acid composition #1: the result of this dilution was a clear solution; in acid composition #2: the result of this dilution was a clear solution; and in Acid composition #3: the result of this dilution was a turbid solution.

Upon visual analysis of the above series of dilutions, it is clear that the surfactant mixture provides satisfactory stability for all the tested dilution scenarios with the exception of Acid composition #3.

In another series of tests, the anionic surfactant was kept as Dowfax® C10L. However, the nonionic surfactant was changed to Novel® 23E3 which has only 3 mol % EO. As shown in table 11, low hand sanitizer concentrations (5 and 10 wt %) could not solubilize any citral. For the composition containing 15 wt % hand sanitizer, the maximum solubilization of citral decreased to 12 wt % compared to 15 wt % for Novel® 23E7. As the concentration of hand sanitizer increases to 20% and 25 wt %, all of the citral added was solubilized into clear transparent solutions.

TABLE 11
Formulations containing citral and a varying amount of hand sanitizer using
DOWFAX ^{®} 10CL and Novel ^{®} 23E3 as surfactant mixture and comments on
the visual aspect of each prepared composition

| | HS-ME51 | | HS-ME52 | | HS-ME53 | | HS-ME54 | | HS-ME55 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| Hand sanitizer | 5.0 | 0.75 | 10.0 | 1.50 | 15.0 | 2.25 | 20.0 | 3.00 | 25.0 | 3.75 |
| Dowfax ^{®} C10L | 10.0 | 1.50 | 10.0 | 1.50 | 10.0 | 1.50 | 10.0 | 1.50 | 10.0 | 1.50 |
| Novel ^{®} 23E3 | 20.0 | 3.00 | 20.0 | 3.00 | 20.0 | 3.00 | 20.0 | 3.00 | 20.0 | 3.00 |
| Citral | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | X | T | X | T | Y | C | Y | C | Y | C |
| 0.90 | | | | | Y | C | Y | C | Y | C |
| 1.35 | | | | | Y | C | Y | C | Y | C |
| 1.80 | | | | | Y | C | Y | C | Y | C |
| 2.25 | | | | | Y | T | Y | C | Y | C |

A photograph was taken of composition HS-ME55 (2 gpt) diluted in various media and resulted in the following: in DIW (the resulting dilution was a clear Solution); in 2% KCl (the result of the dilution was a turbid solution); in acid composition #1 (the resulting dilution was a turbid solution); in acid composition #2 (the resulting dilution was a clear solution); and in Acid composition #4 (the result of the dilution was a turbid solution).

Upon visual analysis of the above series of dilutions, it is clear that the surfactant mixture provides satisfactory stability only for the tested dilution scenario with Acid composition #2.

In another series of testing, the anionic surfactant was changed to DDBSA. However, the nonionic surfactant is kept as Novel® 23E3 which has only 3 mol % EO. As shown in table 12, low hand sanitizer concentrations (5, 10, and even 15 wt %) could not solubilize more than 3 wt % citral. For the compositions containing 20 and 25 wt % of hand sanitizer, all of the citral added was solubilized into clear transparent solutions.

TABLE 12
Formulations containing citral and a varying amount of hand sanitizer using
DDBSA and Novel ^{®} 23E3 as surfactant mixture and comments on the visual
aspect of each prepared composition

| | HS-ME56 | | HS-ME57 | | HS-ME58 | | HS-ME59 | | HS-ME60 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| HS | 5.0 | 0.75 | 10.0 | 1.50 | 15.0 | 2.25 | 20.0 | 3.00 | 25.0 | 3.75 |
| DDBSA | 10.0 | 1.50 | 10.0 | 1.50 | 10.0 | 1.50 | 10.0 | 1.50 | 10.0 | 1.50 |
| Novel ^{®} 23E3 | 20.0 | 3.00 | 20.0 | 3.00 | 20.0 | 3.00 | 20.0 | 3.00 | 20.0 | 3.00 |
| Citral | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | X | VV | X | VV | Y | C/V | Y | C | Y | C |
| 0.90 | | | | | Y | T | Y | C | Y | C |
| 1.35 | | | | | | | Y | C | Y | C |
| 1.80 | | | | | | | Y | C | Y | C |
| 2.25 | | | | | | | Y | C | Y | C |

A photograph was taken of the diluted composition HS-ME60 (2 gpt) in various media resulted in the following: in DIW (the resulting dilution was a clear solution); in 2% KCl (the resulting dilution was a turbid solution); in acid composition #1 the resulting dilution was a turbid solution); in acid composition #2 (the resulting dilution was a turbid solution); and in Acid composition #3 (the resulting dilution was a clear solution).

This confirms that the surfactant mixture is only good for the tested dilution scenarios with Acid composition #3. However, this is the only formulation that works for Acid composition #3.

Nonionic/Nonionic Surfactant Mixture

A mixture of nonionic/nonionic surfactants is tested for solubilization of 15 wt % Citral by varying the concentration of hand sanitizer. One surfactant is Novel® 23E7 which is a linear ethoxylate and the other is Lutensol® XL90 which is branched ethoxylate. As shown in table 13, low hand sanitizer concentrations (5 wt %) can solubilize maximum 6 wt % citral. As the concentration of hand sanitizer increases to 10% and even up to 25 wt %, all of the citral added was solubilized into clear transparent solutions.

A photograph taken showed the composition HS-ME40 (2 gpt) diluted in various media resulted in the following: in DIW: (the resulting dilution was a clear solution); in 2% KCl: (the resulting dilution was a clear solution); in acid composition #1: (the resulting dilution was a clear solution); in acid composition #2: (the resulting dilution was a clear solution); and in Acid composition #3: (the resulting dilution was a turbid solution).

This confirms that the surfactant mixture was stable for all the tested dilution scenarios except for Acid composition #3.

TABLE 13
Formulations containing citral and a varying amount of hand sanitizer using
Lutensol XL90 ^{®} and Novel ^{®} 23E7 as surfactant mixture and comments on the
visual aspect of each prepared composition

| | HS-ME36 | | HS-ME37 | | HS-ME38 | | HS-ME39 | | HS-ME40 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| hand sanitizer | 5.0 | 0.75 | 10.0 | 1.50 | 15.0 | 2.25 | 20.0 | 3.00 | 25.0 | 3.75 |
| Lutensol XL90 ^{®} | 10.0 | 1.50 | 10.0 | 1.50 | 10.0 | 1.50 | 10.0 | 1.50 | 10.0 | 1.50 |
| Novel ^{®} 23E7 | 20.0 | 3.00 | 20.0 | 3.00 | 20.0 | 3.00 | 20.0 | 3.00 | 20.0 | 3.00 |
| Citral | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | Y | C/V | Y | C | Y | C | Y | C | Y | C |
| 0.90 | Y | C | Y | C | Y | C | Y | C | Y | C |
| 1.35 | Y | T | Y | C | Y | C | Y | C | Y | C |
| 1.80 | | | Y | C | Y | C | Y | C | Y | C |
| 2.25 | | | Y | C | Y | C | Y | C | Y | C |

TABLE 14
Particle size distribution of formulations
HS-ME3, HS-ME4 and HS-ME5 as
determined by dynamic light scattering

| | Cumulant | | | | |
|---|---|---|---|---|---|
| | Diameter | | | | |
| Sample | (nm) | PI | D10 | D50 | D90 |
| HS-ME3 | 11.3 | 0.064 | 3.4 | 12.8 | 45.7 |
| HS-ME4 | 13 | 0.129 | 3.8 | 12.7 | 43.7 |
| HS-ME5 | 12.9 | 0.06 | 6.3 | 13.1 | 26.4 |

### Testing with 15 wt % Oil (Mineral Oil)

Nonionic/Anionic Surfactant Mixture

Another experiment studied the solubilization of mineral oil in a nonionic/anionic surfactant mixture of DDBSA and Novel® 23E7. However, this particular surfactant mixture was not good enough for mineral oil despite the fact that it had worked very well for citral.

TABLE 15
Formulations containing mineral oil and a varying amount of hand sanitizer
using DDBSA and Novel ^{®} 23E7 as surfactant mixture and comments on the
visual aspect of each prepared composition

| | HS-ME6 | | HS-ME7 | | HS-ME8 | | HS-ME9 | | HS-ME10 | |
|---|---|---|---|---|---|---|---|---|---|---|
| | wt % | g | wt % | g | wt % | g | wt % | g | wt % | g |
| Water | 50.0 | 7.5 | 45.0 | 6.8 | 40.0 | 6.0 | 35.0 | 5.3 | 30.0 | 4.5 |
| hand sanitizer | 5.0 | 0.8 | 10.0 | 1.5 | 15.0 | 2.3 | 20.0 | 3.0 | 25.0 | 3.8 |
| DDBSA | 10.0 | 1.5 | 10.0 | 1.5 | 10.0 | 1.5 | 10.0 | 1.5 | 10.0 | 1.5 |
| Novel ^{®} 23E7 | 20.0 | 3.0 | 20.0 | 3.0 | 20.0 | 3.0 | 20.0 | 3.0 | 20.0 | 3.0 |
| Mineral Oil | 15.0 | 2.3 | 15.0 | 2.3 | 15.0 | 2.3 | 15.0 | 2.3 | 15.0 | 2.3 |
| Total | 100.0 | 15.0 | 100.0 | 15.0 | 100.0 | 15.0 | 100.0 | 15.0 | 100.0 | 15.0 |
| Mineral Oil (g) | | | | | | | | | | |
| 0.45 | Y | T | Y | C | Y | C | Y | C | Y | T |
| 0.90 | X | | Y | T | Y | T | Y | T | | |
| 1.35 | | | | | | | | | | |
| 1.80 | | | | | | | | | | |
| 2.25 | | | | | | | | | | |

Nonionic/Nonionic Surfactant Mixture

Another experiment studied the solubilization of mineral oil in a nonionic/nonionic surfactant mixture of Lutensol XL90® and Novel® 23E7. However, this surfactant mixture was not good for mineral oil despite the fact that it worked very well for the solubilization of citral.

TABLE 16
Formulations containing mineral oil and a varying amount of hand sanitizer
using XL90 and Novel 23E7 as surfactant mixture and comments on the visual
aspect of each prepared composition

| | HS-ME41 | | HS-ME42 | | HS-ME43 | | HS-ME44 | | HS-ME45 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| HS | 5.0 | 0.75 | 10.0 | 1.50 | 15.0 | 2.25 | 20.0 | 3.00 | 25.0 | 3.75 |
| Lutensol XL90 ^{®} | 10.0 | 1.50 | 10.0 | 1.50 | 10.0 | 1.50 | 10.0 | 1.50 | 10.0 | 1.50 |
| Novel ^{®} 23E7 | 20.0 | 3.00 | 20.0 | 3.00 | 20.0 | 3.00 | 20.0 | 3.00 | 20.0 | 3.00 |
| Mineral Oil | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Mineral Oil (g) | | | | | | | | | | |
| 0.45 | Y | T | Y | T | Y | T | Y | T | Y | T |
| 0.90 | X | | X | | X | | X | | X | |
| 1.35 | | | | | | | | | | |
| 1.80 | | | | | | | | | | |
| 2.25 | | | | | | | | | | |

Novel® 23E7 was replaced by Novel® 23E3 which is a more hydrophobic surfactant as it only contains 3 mol % EO. As shown in the table below, low hand sanitizer concentrations (5 and 10 wt %) can solubilize maximum 3 wt % mineral oil and yields a very viscous solution. As the concentration of hand sanitizer increases to 10 wt %, all of the citral added was solubilized into clear transparent solutions. However, as the concentration of hand sanitizer was increased to 20 and 25 wt %, the solubilization of mineral oil decreased to 12 and 9 wt %, respectively.

A photograph was taken of the diluted HS-ME40 (2 gpt) in DIW (the resulting dilution forms a turbid solution); 2% KCl (the resulting dilution forms a turbid solution); acid composition #1 (the resulting dilution forms a clear solution); acid composition #2 (the resulting dilution forms a clear solution); and Acid composition #4 (the resulting dilution forms a turbid solution).

This confirms that the surfactant mixture is only good for the tested dilution in the scenarios with acid composition #1 and acid composition #2.

TABLE 17
Formutations containing mineral oil and a varying amount of hand sanitizer
using Lutensol XL90 ^{®} and Novel ^{®} 23E7 as surfactant mixture and comments
on the visual aspect of each prepared composition

| | HS-ME46 | | HS-ME47 | | HS-ME48 | | HS-ME49 | | HS-ME50 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| hand sanitizer | 5.0 | 0.75 | 10.0 | 1.50 | 15.0 | 2.25 | 20.0 | 3.00 | 25.0 | 3.75 |
| Lutensol XL90 ^{®} | 10.0 | 1.50 | 10.0 | 1.50 | 10.0 | 1.50 | 10.0 | 1.50 | 10.0 | 1.50 |
| Novel ^{®} 23E3 | 20.0 | 3.00 | 20.0 | 3.00 | 20.0 | 3.00 | 20.0 | 3.00 | 20.0 | 3.00 |
| Mineral Oil | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Mineral Oil (g) | | | | | | | | | | |
| 0.45 | X | VV | X | VV | Y | C | Y | C | Y | C |
| 0.90 | | | | | Y | C | Y | C | Y | C |
| 1.35 | | | | | Y | C | Y | C | Y | T |
| 1.80 | | | | | Y | C | Y | T | | |
| 2.25 | | | | | Y | C | | | | |

Conclusions

From the above experiments, it can be concluded that recycled hand sanitizer can effectively form microemulsions comparable to butyl carbitol.

Microemulsions with mixtures of nonionic/anionic surfactants or nonionic/nonionic surfactants are made and can fit different dilution fluids such as purified water, 2% KCl solutions, 100% acid composition #1, 100% acid composition #2, 100% acid composition #3 or 100% acid composition #4. These microemulsions can be used for numerous applications for acid and nonacid-based solutions for wellbore cleaning.

### Testing HS Gel with 15 wt % Oil (Citral)

Nonionic/Anionic Surfactant Mixture

Hand sanitizers containing gel are typically made using a carbomer. This type of commercial hand sanitizer gel requires special handling to prevent its flocculation and precipitation.

A series of tests were carried out to incorporate hand sanitizer gel into a composition to form a microemulsion. Successfully achieving a stable microemulsion would confirm that even hand sanitizer gel could be employed to prepare microemulsions for uses as mentioned herein. Further, this would confirm the ability to recycle not only non-gel hand sanitizer bit gel-based hand sanitizer as well.

With a DDBSA/Novel® 23E7 combination, the surfactant solutions (before adding Citral) were clear one-phase solutions. A photograph was taken of the microemulsion samples with different concentrations of hand sanitizer gel and a mixture of DDBSA and Novel 23E7 as per the compositions listed in Table 18. It was demonstrated that the various compositions (set out in Table 18) formed microemulsions with hand sanitizer gel and a surfactant mixture of DDBSA and Novel® 23E7. The microemulsions were clear, transparent and non-viscous. However, the composition labelled THSG-ME18 developed high viscosity after a week.

TABLE 18
Formulations containing citral and a varying amount of hand sanitizer (gel)
using DDBSA and Novel ^{®} 23E7 as surfactant mixture and comments on the
visual aspect of each prepared composition

| | THSG-ME16 | | THSG-ME17 | | THSG-ME18 | | THSG-ME1 9 | | THSG-ME20 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| THS-Gel | 5.0 | 0.75 | 10.0 | 1.50 | 15.0 | 2.25 | 20.0 | 3.00 | 25.0 | 3.75 |
| DDBSA | 10.0 | 1.50 | 10.0 | 1.50 | 10.0 | 1.50 | 10.0 | 1.50 | 10.0 | 1.50 |
| Novel ^{®} 23E7 | 20.0 | 3.00 | 20.0 | 3.00 | 20.0 | 3.00 | 20.0 | 3.00 | 20.0 | 3.00 |
| Citral | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | Y | T | Y | C | Y | C | Y | C | Y | C |
| 0.90 | | | Y | T | Y | C | Y | C | Y | C |
| 1.35 | | | | | Y | C | Y | C | Y | C |
| 1.80 | | | | | Y | C | Y | C | Y | C |
| 2.25 | | | | | Y | C | Y | C | Y | C |

Another series of testing investigated another anionic surfactant, Dowfax® C10L in combination with Novel® 23E7 and different concentrations of hand sanitizer gel for various compositions set out in Table 19. A photograph was taken of the surfactant solutions with different concentrations of hand sanitizer gel and a mixture of Dowfax® C10L and Novel® 23E7. The behavior of Dowfax® C10L (FIG. 12) was completely different from that of DDBSA. After adding Dowfax® C10L, at low concentration of hand sanitizer gel, there was flocculation and precipitation of the carbomer. At intermediate concentrations of hand sanitizer gel, there was no flocculation and the solution was clear and transparent. At high concentrations of hand sanitizer gel, there was heavy flocculation and turbidity in the solution.

TABLE 19
Formutations containing citral and a varying amount of hand sanitizer (gel)
using Dowfax ^{®} C10L and Novel ^{®} 23E7 as surfactant mixture and comments
on the visual aspect of each prepared composition

| | THSG-ME21 | | THSG-ME22 | | THSG-ME23 | | THSG-ME24 | | THSG-ME25 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| THS-Gel | 5.0 | 0.75 | 10.0 | 1.50 | 15.0 | 2.25 | 20.0 | 3.00 | 25.0 | 3.75 |
| Dowfax ^{®} C10L | 10.0 | 1.50 | 10.0 | 1.50 | 10.0 | 1.50 | 10.0 | 1.50 | 10.0 | 1.50 |
| Novel ^{®} 23E7 | 20.0 | 3.00 | 20.0 | 3.00 | 20.0 | 3.00 | 20.0 | 3.00 | 20.0 | 3.00 |
| Citral | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | X | | X | | Y | C/F | Y | C/F | Y | C/F |
| 0.90 | | | | | Y | C/F | Y | C/F | Y | C/F |
| 1.35 | | | | | Y | C/F | Y | C/F | Y | C/F |
| 1.80 | | | | | Y | C/F | Y | C/F | Y | C/F |
| 2.25 | | | | | Y | C/F | Y | C/F | Y | C/F |

After adding citral to the composition labelled THSG-ME23-25, a microemulsion was formed for each one of the compositions. The microemulsion was a very clear and transparent solution, regardless of carbomer precipitation at the bottom of the solution vials.

A photograph was taken of the microemulsions with different concentrations of THS (hand sanitizer) gel and mixture of Dowfax® C10L and Novel® 23E7.

Nonionic/Nonionic Surfactant Mixture

A mixture of two nonionic surfactants, Lutensol® XL90 and Novel 23E7, was tested. Formulations with different concentrations of hand sanitizer gel and two different ratios of Lutensol® XL90 and Novel® 23E7 were made. Solutions were clear and transparent after adding Lutensol® XL90, and then became slightly hazy after adding Novel® 23E7.

A photograph was taken of the microemulsions with different concentrations of hand sanitizer gel and a mixture of Lutensol® XL90 and Novel® 23E7 (10:20). After adding citral, the microemulsion was formed, however, it maintained the same haziness as the surfactant solution.

When the composition labelled THSG-ME5 microemulsion was diluted in different media, initially the diluted compositions formed clear solutions in DIW, 2% KCl, acid composition #1, Acid composition #3, but were turbid in Acid composition #4. After a few days, the acid dilutions presented flocculation and precipitation, while the KCl dilution remained a clear and transparent solution.

TABLE 20
Formulations containing citral and a varying amount of hand sanitizer (gel)
using Lutensol ^{®} XL90 and Novel ^{®} 23E7 as surfactant mixture and comments
on the visual aspect of each prepared composition

| | THSG-ME1 | | THSG-ME2 | | THSG-ME3 | | THSG-ME4 | | THSG-ME5 | | THSG-ME6 | |
|---|---|---|---|---|---|---|---|---|---|---|---|---|
| | wt % | g | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 | 25.0 | 3.75 |
| THS-Gel | 5.0 | 0.75 | 10.0 | 1.50 | 15.0 | 2.25 | 20.0 | 3.00 | 25.0 | 3.75 | 30.0 | 4.50 |
| Lutensol ^{®} | 10.0 | 1.50 | 10.0 | 1.50 | 10.0 | 1.50 | 10.0 | 1.50 | 10.0 | 1.50 | 10.0 | 1.50 |
| XL90 | | | | | | | | | | | | |
| Novel ^{®} | 20.0 | 3.00 | 20.0 | 3.00 | 20.0 | 3.00 | 20.0 | 3.00 | 20.0 | 3.00 | 20.0 | 3.00 |
| 23E7 | | | | | | | | | | | | |
| Citral | 15.0 | 2.25 | 15.0 | 2.25 | 15.0 | 2.25 | 15.0 | 2.25 | 15.0 | 2.25 | 15.0 | 2.25 |
| Total | 100 | 15.0 | 100 | 15.0 | 100 | 15.0 | 100 | 15.0 | 100 | 15.0 | 100 | 15.0 |
| Citral (g) | | | | | | | | | | | | |
| 0.45 | Y | Hazy/ | Y | Hazy/ | Y | Hazy/ | Y | Hazy/ | Y | Hazy/ | Y | Hazy/ |
| | | No | | No | | No | | No | | No | | No |
| | | Sep | | Sep | | Sep | | Sep | | Sep | | Sep |
| 0.90 | Y | ~ | Y | ~ | Y | ~ | Y | ~ | Y | ~ | Y | ~ |
| 1.35 | Y | T | Y | ~ | Y | ~ | Y | ~ | Y | ~ | Y | ~ |
| 1.80 | | | Y | ~ | Y | ~ | Y | ~ | Y | ~ | Y | ~ |
| 2.25 | | | Y | ~ | Y | ~ | Y | ~ | Y | ~ | Y | ~ |

A photograph was taken of the diluted microemulsions with different concentrations of hand sanitizer gel and a mixture of Lutensol® XL90 and Novel® 23E7 (10:20). The composition labelled THSG-ME5 (2 gpt) was diluted in the following media: in DIW (the result of the dilution was a clear solution); in 2% KCl (the result of the dilution was a clear solution); in acid composition #1 (the result of the dilution was a clear solution); in acid composition #2 (the result of the dilution was a clear solution); and in Acid composition #4 (the result of the dilution was a turbid solution).

This confirms that the surfactant mixture performed well in all of the tested dilution scenarios with the exception of Acid composition #3.

TABLE 21
Formulations containing citral and a varying amount of hand sanitizer (gel)
using Lutensol ^{®} XL90 and Novel ^{®} 23E7 as surfactant mixture and comments
on the visual aspect of each prepared composition

| | THSG-ME7 | | THSG-ME8 | | THSG-ME9 | | THSG-ME10 | | THSG-ME11 | |
|---|---|---|---|---|---|---|---|---|---|---|
| | wt % | g | wt % | g | wt % | g | wt % | g | wt % | g |
| Water | 50.0 | 7.50 | 45.0 | 6.75 | 40.0 | 6.00 | 35.0 | 5.25 | 30.0 | 4.50 |
| THS-Gel | 5.0 | 0.75 | 10.0 | 1.50 | 15.0 | 2.25 | 20.0 | 3.00 | 25.0 | 3.75 |
| Lutensol ^{®} | 15.0 | 2.25 | 15.0 | 2.25 | 15.0 | 2.25 | 15.0 | 2.25 | 15.0 | 2.25 |
| XL90 | | | | | | | | | | |
| Novel ^{®} 23E7 | 15.0 | 2.25 | 15.0 | 2.25 | 15.0 | 2.25 | 15.0 | 2.25 | 15.0 | 2.25 |
| Citral | 15.0 | 2.25 | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 | 100.0 | 15.0 |
| Citral (g) | | | | | | | | | | |
| 0.45 | Y | Hazy/ | Y | Hazy/No | Y | Hazy/ | Y | Hazy/ | Y | Hazy/No |
| | | No Sep | | Sep | | No Sep | | No Sep | | Sep |
| 0.90 | Y | ~ | Y | ~ | Y | ~ | Y | ~ | Y | ~ |
| 1.35 | Y | T | Y | ~ | Y | ~ | Y | ~ | Y | ~ |
| 1.80 | | | Y | ~ | Y | ~ | Y | ~ | Y | ~ |
| 2.25 | | | Y | ~ | Y | ~ | Y | ~ | Y | ~ |

A photograph was taken of the diluted microemulsions with different concentrations of THS (hand sanitizer) gel and mixture of Lutensol® XL90 and Novel® 23E7 (15:15). The composition labelled THSG-ME5 (2 gpt) was diluted in the following media: in DIW (the result of the dilution was a turbid solution); in 2% KCl (the result of the dilution was a clear solution); in acid composition #1 (the result of the dilution was a clear solution); in acid composition #2 (the result of the dilution was a clear solution); and in Acid composition #3 (the result of the dilution was a turbid solution).

This confirms that the surfactant mixture performed well in all of the tested dilution scenarios with the exception of DIW and Acid composition #3.

### Testing Protectorplus® Gel with 15 wt % Oil

### Citral

Nonionic/Nonionic Surfactant Mixture

Commercial hand sanitizer gel, ProtectorPlus® Gel, was also tested for its effectiveness in forming microemulsion. The major component of this gel is carbomer. In general, the behavior is similar to THS (hand sanitizer) gel (FIG. 18). A photograph was taken of the diluted microemulsions with different concentrations of ProtectorPlus® Gel and a mixture of Lutensol® XL90 and Novel® 23E7 (10:20). After adding Novel® 23E7, the solution was hazy, and when citral was added microemulsion formed but maintained the same haziness as the surfactant solution.

TABLE 22
Formulations containing citral and a varying amount of hand sanitizer (gel)
using Lutensol ^{®} XL90 and Novel ^{®} 23E7 as surfactant mixture and comments
on the visual aspect of each prepared composition

| | THSG-ME12 | | THSG-ME13 | | THSG-ME14 | | THSG-ME15 | |
|---|---|---|---|---|---|---|---|---|
| | wt % | g | wt % | g | wt % | g | wt % | g |
| Water | 45.0 | 6.75 | 40.0 | 6.00 | 35.0 | 5.25 | 30.0 | 4.50 |
| ProtectorPlus-Gel | 10.0 | 1.50 | 15.0 | 2.25 | 20.0 | 3.00 | 25.0 | 3.75 |
| Lutensol ^{®} XL90 | 10.0 | 1.50 | 10.0 | 1.50 | 10.0 | 1.50 | 10.0 | 1.50 |
| Novel ^{®} 23E7 | 20.0 | 3.00 | 20.0 | 3.00 | 20.0 | 3.00 | 20.0 | 3.00 |
| Citral | 15.0 | 2.25 | 15.0 | 2.25 | 15.0 | 2.25 | 15.0 | 2.25 |
| Total | 100.0 | 15.0 | 100.0 | 15.0 | 100.0 | 15.0 | 100.0 | 15.0 |
| Citral (g) | | | | | | | | |
| 0.45 | Y | Hazy/No | Y | Hazy/No | Y | Hazy/Free | Y | Hazy/No |
| | | Sep | | Sep | | Gel | | Sep |
| 0.90 | Y | ~ | Y | ~ | Y | ~ | Y | ~ |
| 1.35 | Y | ~ | Y | ~ | Y | ~ | Y | ~ |
| 1.80 | Y | ~ | Y | ~ | Y | ~ | Y | ~ |
| 2.25 | Y | ~ | Y | ~ | Y | ~ | Y | ~ |

The observations from the above experiments confirm that both hand sanitizer and hand sanitizer gel can effectively form microemulsions comparable to butyl carbitol. It is to be noted that carbomers in hand sanitizer gel and other commercial hand sanitizer gels needs special handling to prevent its flocculation and precipitation.

Microemulsions with mixtures of nonionic/anionic surfactants or nonionic/nonionic surfactants are made and can fit different dilution fluids such as purified water, 2% KCl solutions, 100% acid composition #1, 100% acid composition #2, 100% acid composition #3 or 100% acid composition #4.

According to a preferred embodiment of the present invention, these microemulsions can be used for numerous applications for acid and non-acid-based solutions for wellbore cleaning.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A microemulsion composition comprising:
a hand sanitizer product in an amount ranging from 10 to 40 wt % of the total weight of the composition, said hand sanitizer product comprising an alcohol selected from the group consisting of: ethanol and isopropanol, wherein said alcohol is present in an amount ranging from 60 to 85 wt %;
oil phase in an amount ranging from 10 to 40 wt % of the total weight of the composition;
a nonionic surfactant present in an amount ranging from 10 to 30 wt % of the total weight of the composition;
an anionic surfactant present in an amount ranging from 5 to 15 wt % of the total weight of the composition; and
water present in an amount ranging from 30 to 70 wt % of the total weight of the composition.

2. The microemulsion according to claim 1, where the oil phase is selected from the group consisting of: citral, terpenes, hydrocarbon oils, methyl esters; and combinations thereof.

3. The microemulsion according to claim 1, where the oil phase is citral.

4. The microemulsion according to claim 1, where the oil phase is present in an amount ranging from 20 to 35 wt % of the total weight of the composition.

5. The microemulsion according to claim 1, where the oil phase is present in an amount ranging from 25 to 30 wt % of the total weight of the composition.

6. The microemulsion according to claim 1, where the nonionic surfactant is selected from the group consisting of nonionic linear or branched alcohol ethoxylate.

7. The microemulsion according to claim 1, where the nonionic surfactant present in an amount ranging from 15 to 25 wt % of the total weight of the composition.

8. The microemulsion according to claim 1, where the nonionic surfactant present in an amount of approximately 20 wt % of the total weight of the composition.

9. The microemulsion according to claim 1, where the anionic surfactant present in an amount ranging from 7 to 13 wt % of the total weight of the composition.

10. The microemulsion according to claim 1, where the anionic surfactant present in an amount of approximately 10 wt % of the total weight of the composition.

11. The microemulsion according to claim 1, where the anionic surfactant is selected from the group consisting of: anionic sulfonate surfactant.

12. The microemulsion according to claim 11, where the anionic surfactant is selected from the group consisting of: DDBSA; Dowfax® C10L; and combinations thereof.

13. The microemulsion according to claim 1, where the nonionic surfactant is selected from the group consisting of: Novel® 23E3; Novel® 23E7; Lutensol® XL90; and combinations thereof.

14. The microemulsion according to claim 1, further comprising a carbomer.

15. The microemulsion according to claim 14, wherein said carbomer is present in a concentration ranging from 0.1 to 5 wt % of the total weight of the microemulsion.

16. The microemulsion according to claim 1, wherein said microemulsion is stable for a period of at least 7 days at room temperature.

17. Method of recycling an expired hand sanitizer product containing ethanol in an amount ranging from 60 to 85 wt %, wherein said method comprises:
providing said hand sanitizer product;
combining said hand sanitizer product with an anionic surfactant;
adding a nonionic surfactant to the resulting mixture; and
adding citral to the mixture containing said hand sanitizer, nonionic surfactant and said anionic surfactant to create a stable microemulsion;
wherein said stable microemulsion can be further diluted with an acidic composition to be used in industrial applications comprising:
flowback aids for hydraulic fracturing in tight reservoirs and unconventional shale reservoirs;
naphthenate deposition control using acids/alcohols mixtures; or
water-in-oil demulsifier using ethanol for bitumen emulsions from oilsand.

18. Use of a microemulsions according to claim 1 in combination with a modified acid microemulsion-based dissolver to remove of mixed scale deposition.

19. Use of a microemulsions according to claim 1 in combination with a modified acid as a microemulsion-based dissolver for cleaning of surface facilities.

20. Use of a microemulsions according to claim 1 in combination with a modified acid as a microemulsion-based dissolver cleanup of wellbore deposits concentrated with paraffin and asphaltene.

21. Use of a microemulsions according to claim 1 in combination with a modified acid microemulsion-based dissolver for filter cake removal.
